(19) United States
(12) Patent Application Publication
(10) Pub. No.: US 2023/0218514 A1
(43) Pub. Date: Jul. 13, 2023

(54) FLOATING SUSTAINED RELEASE TABLET FORMULATION OF DROXIDOPA AND METHOD THEREOF

(71) Applicants: VARALAKSHMI MUMMIDI, Kakinada (IN); NARASIMHA SAI SHANMUKHA TALLAPRAGADA, Kakinada (CN); ALLURI GOPALA KRISHNA, Kakinada (IN); GOTTIPATI PADMAJA RANI, Kakinada (IN); AJAY BABU CHIRUMELLA, Narasarao Peta (IN); SANDHYA MADHURI MADDALA, Kakinada (IN)

(72) Inventors: VARALAKSHMI MUMMIDI, Kakinada (IN); NARASIMHA SAI SHANMUKHA TALLAPRAGADA, Kakinada (IN); ALLURI GOPALA KRISHNA, Kakinada (IN); GOTTIPATI PADMAJA RANI, Kakinada (IN); AJAY BABU CHIRUMELLA, Narasarao Peta (IN); SANDHYA MADHURI MADDALA, Kakinada (IN)

(21) Appl. No.: 18/096,046

(22) Filed: Jan. 12, 2023

(30) Foreign Application Priority Data
Jan. 13, 2022 (IN) 202241002161

(51) Int. Cl. A61K 9/00; A61K 31/198; A61K 9/20

(52) U.S. Cl. CPC A61K 9/0065; A61K 31/198; A61K 9/2013; A61K 9/2009; A61K 9/2054

(57) ABSTRACT

The present invention to unit dosage forms which are capable of floating on gastric fluid and delivering the therapeutic agent over an extended period of time. Particularly the invention relates to Droxidopa sustained release gastroretentive dosage form for release of drug over period of 12 hrs. The gastroretentive dosage form of Droxidopa of the present invention comprises drug, polymer (Hydroxy propyl methyl cellulose K200M) as rate controlling polymer and the formulation maintains buoyancy on the gastric fluid for a period of 12 hrs, thus providing better patient compliance by providing reducing dosing frequency.

### FIELD OF INVENTION

This invention relates to therapeutic compositions in unit dosage form which are capable of floating on gastric fluid and delivering their contained therapeutic agent over an extended period of time. Particularly the invention relates to sustained release Droxidopa tablets, particularly floating tablets of Droxidopa. The invention relates to gastro-retentive drug delivery system, wherein the tablets float on gastric fluid and deliver the drug for sustained time period.

### BACKGROUND OF INVENTION

Droxidopa is a synthetic amino acid precursor which acts as a prodrug to the neurotransmitters norepinephrine (noradrenaline) and epinephrine (adrenaline) and which is used to increase the concentrations of these neurotransmitters in the body and brain. Chemically, droxidopa is (−)-threo-3-(3,4-Dihydroxyphenyl)-L-serine.

The droxidopa dosage form currently approved for use in the United States is immediate release oral capsules comprising 100, 200, or 300 mg droxidopa, with a maximum daily dosage limited to 1800 mg. Current dosing regimens are administration of one or two capsules three times daily.

US2019321318 discloses oral pharmaceutical compositions which include an extended-release multi-particulate comprising an effective amount of droxidopa, or a pharmaceutically acceptable salt thereof, and a release-controlling agent are disclosed. It is a minitablet or a multiparticulate dosage form preferably a tablet or capsule with a first extended release layer which is lipophilic and second layer with enteric coating. It also discloses an extended release suspension.

U.S. Pat. No. 8,383,681B2 invention provides pharmaceutical compositions useful in the treatment of various conditions or disorders. It comprisesdroxidopa alone or in combination with one or more further pharmaceutically active compounds. The invention further provides methods of treating a variety of conditions or disorders. The invention is directed to a method of treating a condition comprising administering to a subject in need of treatment of the condition a pharmaceutical composition comprising a therapeutically effective amount of droxidopa or a pharmaceutically acceptable ester, amide, salt, solvate, prodrug, or isomer thereof, wherein the condition is selected from the group consisting of a mood disorder, a sleep.

EP2142185A2 discloses a pharmaceutical compositions comprising Droxidopa alone, or in combination with one or more further active ingredients, that can be administered to a patient for the treatment of neurally mediated hypotension.

CN104434868A discloses stable droxidopa oral dosage form facilitating alimentary canal dissolution. The oral dosage form containing Droxidopa, comprises of one or more of a diluent, a binder, a lubricant, a disintegrating agent. It further comprises an antioxidant selected is vitamin VC (L-ascorbic acid), and the ratio of the amount of the active ingredient to the active ingredient of cetopa is 0.05:1 to 1:1. Vitamin c helps in enhancing absorption of the drug by the small intestine by promoting dissolution of the active ingredient, droxidopa, in the digestive tract.

EP3558268A1 discloses droxidopa compositions and methods directed to a pharmaceutical composition comprising droxidopa, or a pharmaceutically acceptable salt thereof, for oral administration, wherein the pharmaceutical composition is an extended release liquid dosage form or solid dosage form. The extended-release multi-particulate comprises an effective amount of droxidopa, or a pharmaceutically acceptable salt thereof, and a release-controlling agent; It may further include a liquid vehicle. The multi-particulate comprises two or more types of pellets, e.g., one type can be immediate release pellets whereas another type can release the drug over an extended period. The liquid vehicle has a pH of less than 7.0. In some embodiments, the liquid vehicle includes a buffering agent.

EP1886678A1 discloses a stable tablet containing Droxidopa. The tablet which is rapidly disintegrated in the oral cavity, comprises droxidopa as an active ingredient, and droxidopa content is 20-80 wt % relative to the total weight of the tablet: mannitol as an excipient, a disintegrant comprising cornstarch or partly pre-gelatinized corn-derived starch, and further comprising corn-derived starch as a binder, wherein the tablet does not contain a binder other than corn-derived starch and the tablet does not contain polyvinylpyrrolidone, hydroxypropylmethylcellulose, hydroxypropylcellulose, agar or gelatin.

EP2363124A1 discloses a pharmaceutical compositions comprising Droxidopa wherein the pharmaceutical composition comprising Droxidopa in combination with one or more additional compounds selected from the group consisting of: entacapone, a catechol-O-methyltransferase inhibitor that is specific for peripheral activity, and provided as a controlled or sustained or delayed release pharmaceutical formulation.

Accordingly, there is a need to improve the solubility, bioavailability and the thus aid the better targeted delivery of the drug.

### OBJECTS OF INVENTION

It is a primary object of the present invention to provide a sustained release Droxidopa floating tablets to provide better patient compliance by providing reducing dosing frequency.

Droxidopa has more stable in gastric acidic pH than in alkaline pH conditions, hence, it is an object of the invention to overcome the instability of Droxidopa in alkaline pH and was formulated Droxidopa as gastro retentive drug delivery as floating tablet, that retains in acidic (gastric) pH for prolonged period of time.

It is yet another object of the present invention to formulate and evaluate sustained release Droxidopa floating tablets by simple and cost effective technique for the treatment of orthostatic hypotension.

It is yet another object of the present invention to provide a floating drug delivery system for Droxidopa that can effectively reduce the dosing frequency by sustained action dosage form.

It is yet another object of the present invention to provide a floating drug delivery system for Droxidopa which increases the patient compliance and effectiveness of therapy.

### SUMMARY OF THE INVENTION

Thus, according to the present invention, there is provided a floating sustained release tablet formulation of Droxidopa, comprising: Droxidopa, controlled release polymer, additives comprising diluent, effervescent agent, gas generating agent, glidant and lubricant, wherein the total tablet weight ranges from 590 to 610 mg and the sustained release tablet is a gastro-retentive tablet that is buoyant in gastric acid and releases Droxidopa in sustained manner for a period of 10-12 hours.

It is another aspect of the present invention to provide a floating sustained release tablet formulation of Droxidopa, wherein the diluent is microcrystalline cellulose.

It is another aspect of the present invention to provide a floating sustained release tablet formulation of Droxidopa, wherein the gas generating agent is sodium bicarbonate.

It is another aspect of the present invention to provide a floating sustained release tablet formulation of Droxidopa, wherein the effervescent agent is anhydrous citric acid or tartaric acid.

It is another aspect of the present invention to provide a floating sustained release tablet formulation of Droxidopa, wherein the glidant is Talc and lubricant is Magnesium stearate.

It is another aspect of the present invention to provide a floating sustained release tablet formulation of Droxidopa, wherein floating lag time of the tabled is less than 60 seconds and buoyancy time of over 12 hrs.

It is another aspect of the present invention to provide a floating sustained release tablet formulation of Droxidopa, wherein the tablet formulation dosage is twice a day.

It is yet another aspect of the present invention to provide a method of preparation of floating sustained release tablet formulation of Droxidopa, comprising steps:
accurately weighing Droxidopa—200-400 mg, polymer HPMCK200M (90-100 mg), sodium bicarbonate 50 mg, citric acid—75 mg, micro crystalline cellulose-115 mg and blending, addition of lubricant-10 mg of magnesium stearate and glidant −10 mg talc added as post lubricant and further mixed for additional 2-3 minutes, sieving of ingredients by passing through 40# sieves; and′ punching of gastro retentive tablets using the rotary tablet compression machine by direct compression technique using 12 mm punches, and wherein the tablets are buoyant on gastric fluid and release Droxidopa for sustained period of over 12 hrs.

### BRIEF DESCRIPTION OF DRAWINGS

The annexed drawings show an embodiment of the present invention, wherein:

FIG. 1: illustrates the DSC Thermogram of Droxidopa

FIG. 2: illustrates the DSC Thermogram of HPMC K200M

FIG. 3: illustrates the DSC Thermogram of Droxidopa with HPMC K 200M

FIG. 4: illustrates the FT-IR spectra of Droxidopa

FIG. 5: illustrates the FT-IR spectra of HPMC K200M

FIG. 6: illustrates the FT-IR spectra of Droxidopa with HPMC K 200M

FIG. 7: illustrates the dissolution profiles of sustained release Droxidopa floating tablet formulations (F1 to F9).

FIG. 8: illustrates the dissolution profiles of sustained release Droxidopa floating tablet formulations (F11 to F18).

FIG. 9: illustrates the Zero order plot of Droxidopa tablet formulations (F1 to F9)

FIG. 10: illustrates the Zero order plot of Droxidopa tablet formulations (F11 to F18)

FIG. 11: illustrates the First order plot of Droxidopa tablet formulations (F1-F9).

FIG. 12: illustrates the First order plot of Droxidopa tablet formulations (F11-F19).

FIG. 13: Higuchi plots of Droxidopa tablet formulations (F1 to F9)

FIG. 14: Higuchi plots of Droxidopa tablet formulations (F11-F18)

FIG. 15: Peppas plots of Droxidopa tablet formulations (F1 to F9)

FIG. 16: Peppas plot of Droxidopa tablet formulations (F11 to F18)

### DETAILED DESCRIPTION OF THE INVENTION ACCOMPANYING FIGURES

Droxidopa is a prodrug of norepinephrine indicated for the treatment of orthostatic hypotension. The purpose of the present invention was to develop a sustained release Floating Drug Delivery System/FDDS of Droxidopa. It has a half-life of 2-3 hrs and belongs to BCS Class III drug. It is a highly unstable in alkaline medium and to overcome this problem, it was fabricated as gastro retentive drug delivery in the form of floating tablets by using hypromellose as rate retarding polymer.

Gastro retentive dosage forms are drug delivery systems which remain in the stomach for an extended period of time and allow both spatial and time control of drug liberation. The aim of the present invention was to develop sustained release Droxidopa floating tablets by using rate retardant polymers, and evaluate different rate retardant polymers and provide a optimized Droxidopa floating tablet. There is currently no formulation of Droxidopa which is controlled release and can be given as twice daily. Currently marketed formulation is a oral capsule 100 mg given thrice daily or t.i.d. novel.

The object of the invention is since, Droxidopa has more stable in gastric acidic pH than in alkaline pH conditions, to overcome the instability of Droxidopa in alkaline pH, it was formulated as floating drug delivery system. The Droxidopa floating tablets reduce the dosing frequency by sustained action dosage form.

Droxidopa

Droxidopa is a prodrug of norepinephrine indicated for the treatment of orthostatic dizziness, lightheadedness, or the “feeling that you are about to black out” in adult patients with symptomatic neurogenic orthostatic hypotension caused by primary autonomic failure including Parkinson disease (PD).

SYNONYMS: threoDOPS, L-DOPS.

IUPAC NAME: (2S,3R)-2-amino-3-(3,4-dihydroxyphenyl)-3-hydroxypropanoic acid; DL-threo-beta-(3,4-Dihydroxyphenyl)serine.

MOLECULAR FORMULA: C₉H₁₁NO₅.

MOLECULAR WEIGHT: 213.189 g/mol

MOLECULAR STRUCTURE OF DROXIDOPA

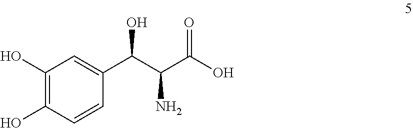 [SMILES: N[C@H](C(=O)O)[C@H](O)c1ccc(O)c(O)c1]

PHYSICO CHEMICAL PROPERTIES:
Colour: White to off white
Density: 1.608 g/cm³
Boiling point: 549.8° C. at 700 mm Hg
Melting point: 232-235° C.
Flash point: 286.3° C.
Solubility: ≥3 mg/ml
Refractive index: 1.692
Vapour pressure: 6.39 E−13 mm hg at 25° C.
Solubility: Slightly soluble in water, Practically insoluble in methanol, glacial acetic acid, ethanol, acetone, chloroform. Soluble in dilute HCl

Mechanism of Action

Droxidopa crosses the blood-brain barrier where it is converted to norepinephrine via decarboxylation by L-aromatic-amino-acid decarb oxylase. Increased levels of norepinephrine in the central nervous system (CNS) may be beneficial to patients in a wide range of indications. Norephinephrine acts at alpha-adrenergic receptors as a vasoconstrictor and at beta-adrenergic receptors as a heart stimulator and artery dilator.

Pharmacokinetic Parameters:

Absorption:
Oral bioavailability is 90%
Cₘₐₓ: 3160 ng/mL
Tₘₐₓ: 1-4 hrs

Distribution:
Volume of distribution=200 L
Protein binding=75%

Metabolism:
Metabolised by L-amino acid decarboxylase
Metabolite is 3-O-methyl dihydroxy phenyl serine

Elimination:
Droxidopa is mainly eliminated through urine.
Elimination half life is 2.5 hrs.

DOSE: 100 mg, 200 mg and 300 mg

Therapeutic Uses:

It is mainly used in the treatment of neuro orthostatic hypotension. It is used in the treatment of Parkinsons disease.

Toxicity Summary

Droxidopa has minimal toxic effects and acute, oral LD50 of more than 5 g/kg in mice, rats, dogs and monkeys. Side effects occur in 0.78% of patients which includes nausea, headache, dizziness, increased B.P, hallucination and anorexia. Hepatotoxicity was not found.

Method of Formulation of Droxidopa Floating Tablets

A sustained release dosage form of Droxidopa was designed as twice daily formulation for oral administration. Eighteen formulations of Droxidopa floating tablets were prepared with varying concentrations of polymers by direct compression technique. The formulations were composed of various grades of hydrophilic polymers, i.e., hypromellose [hydroxyl propyl methylcellulose (HPMC K15M, HPMC K50M HPMC K100M and HPMC K200M)], acrylate polymer (Eudragit L100) for sustained release purpose. Buoyancy was achieved by adding an effervescent mixture of sodium bicarbonate with anhydrous citric acid and floating lag time and total buoyancy time was evaluated. The prepared floating tablets were evaluated for pre and post compression parameters. The in vitro Droxidopa release in 0.1N HCl were subjected to different models in order to evaluate their release kinetics and mechanism. Stability studies were carried out to the optimized formulation as per ICH guidelines.

The process of formulation involved the following steps:

In pre-formulation studies, to perform drug-excipient compatibility studies by different techniques like FTIR and DSC.

Formulation Development

a. Formulation of sustained release floating tablets of Droxidopa with different concentrations of HPMC K15M, HPMC K50M, HPMC K100M, HPMC K200M and EudragitL100 polymers.

b. Preparation of sustained release Droxidopa floating tablets by direct compression method.

Table 1 and 2 illustrates the formulation design of sustained release Droxidopa floating tablets:

Preparation of Droxidopa Sustained Release Floating Tablets:

The eighteen formulations of droxidopa floating tablets were prepared by varying concentrations of polymers as shown in Table 1 and 2 by direct compression technique.

A sustained release dosage form of Droxidopa was designed as twice daily formulation for oral administration. The formulations were composed of various grades of hydrophilic polymers, Hypromellose [(hydroxyl propyl methylcellulose) HPMCK15M, HPMC K100M and HPMC K200M] and a Polyacrylate polymer (Eudragit L100) for gel forming properties. Buoyancy was achieved by adding a gas generating agent and an effervescent agent. In exemplary embodiments the gas generating agent is selected from sodium bicarbonate. The effervescent mixture of sodium bicarbonate and anhydrous citric acid provide the necessary buoyancy upon contact with gastric fluid. It was maintained that the release of the active compound was better controlled when compared with conventional dosage forms with delayed pyloric passage. The gel forming polymers formed a hydrated gel matrix that entrapped the gas, causing the tablet to float and be retained in the stomach or upper part of the small intestine. The hydrated gel matrix created a tortuous path for the drug, resulting in sustained release of the drug.

Accurately weighed the Droxidopa, polymer, sodium bicarbonate, citric acid, micro crystalline cellulose (diluent) and uniformly blended for few minutes. Then, talc (lubricant) and magnesium stearate were added as post lubricant and further mixed for additional 2-3 minutes. All the ingredients were passed through 40# sieves. Then the tablets were prepared using rotary tablet compression machine by direct compression technique using 12 mm punches.

For F10 formulation 500 mg of Eudragit L100 is dissolved in 4 ml methanol; drug is added to it and stirred with glass rod. Now the product is dried and the dried product is compressed into tablet by adding excipients.

TABLE 1
Formulation design of Droxidopasustained
release floating tablets (F1-F10)

| | HPMC | | | HPMC | | | HPMC | | | F10 |
|---|---|---|---|---|---|---|---|---|---|---|
| Ingredients | K 15M | | | K 50M | | | K100M | | | Eudragit |
| (mg) | F1 | F2 | F3 | F4 | F5 | F6 | F7 | F8 | F9 | L100 |
| Droxidopa | 300 | 300 | 300 | 300 | 300 | 300 | 300 | 300 | 300 | 300 |
| Polymer | 50 | 70 | 90 | 50 | 70 | 90 | 50 | 70 | 90 | 500 |
| Micro- | 115 | 90 | 65 | 115 | 90 | 65 | 115 | 90 | 65 | 65 |
| crystalline | | | | | | | | | | |
| cellulose | | | | | | | | | | |
| Citric acid | 75 | 75 | 75 | 75 | 75 | 75 | 75 | 75 | 75 | 75 |
| anhydrous | | | | | | | | | | |
| Sodium bi | 30 | 30 | 30 | 30 | 30 | 30 | 50 | 50 | 50 | 50 |
| carbonate | | | | | | | | | | |
| Talc | 10 | 10 | 10 | 10 | 10 | 10 | 10 | 10 | 10 | 10 |
| Magnesium | 10 | 10 | 10 | 10 | 10 | 10 | 10 | 10 | 10 | 10 |
| stearate | | | | | | | | | | |
| Total tablet | 590 | 585 | 580 | 590 | 585 | 580 | 610 | 605 | 600 | 1010 |
| weight | | | | | | | | | | |

TABLE 2
Formulation design of Droxidopa sustained
releasefloating tablets (F11-F18)

| Ingredients | | | | | | | | |
|---|---|---|---|---|---|---|---|---|
| (mg) | F11 | F12 | F13 | F14 | F15 | F16 | F17 | F18 |
| Droxidopa | 300 | 300 | 300 | 300 | 300 | 300 | 300 | 300 |
| HPMC | 50 | 70 | 90 | 100 | 125 | 150 | 200 | 250 |
| K200M | | | | | | | | |
| Micro | 115 | 90 | 65 | 65 | 40 | 15 | 65 | 15 |
| crystalline | | | | | | | | |
| cellulose | | | | | | | | |
| Citric acid | 75 | 75 | 75 | 75 | 75 | 75 | 75 | 75 |
| anhydrous | | | | | | | | |
| Sodium | 50 | 50 | 50 | 50 | 50 | 50 | 50 | 50 |
| bicarbonate | | | | | | | | |
| Talc | 10 | 10 | 10 | 10 | 10 | 10 | 10 | 10 |
| Magnesium | 10 | 10 | 10 | 10 | 10 | 10 | 10 | 10 |
| stearate | | | | | | | | |
| Total tablet | 610 | 605 | 600 | 610 | 610 | 610 | 710 | 710 |
| weight | | | | | | | | |

Drug—Excipients Compatibility Studies

The present invention discloses the drug excipient compatibility studies as determined by two following methods.

1. Fourier transform infrared spectroscopy (FTIR)

2. Differential scanning calorimeter (DSC)

Differential Scanning Calorimetry (Dsc) Studies

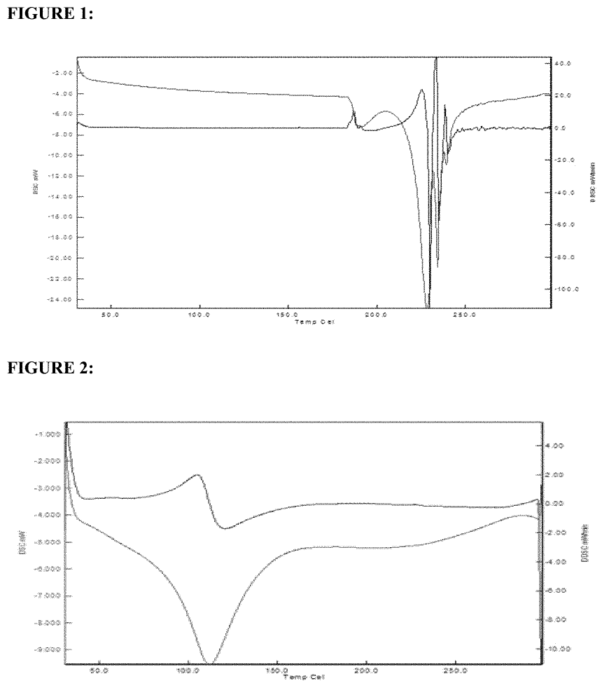

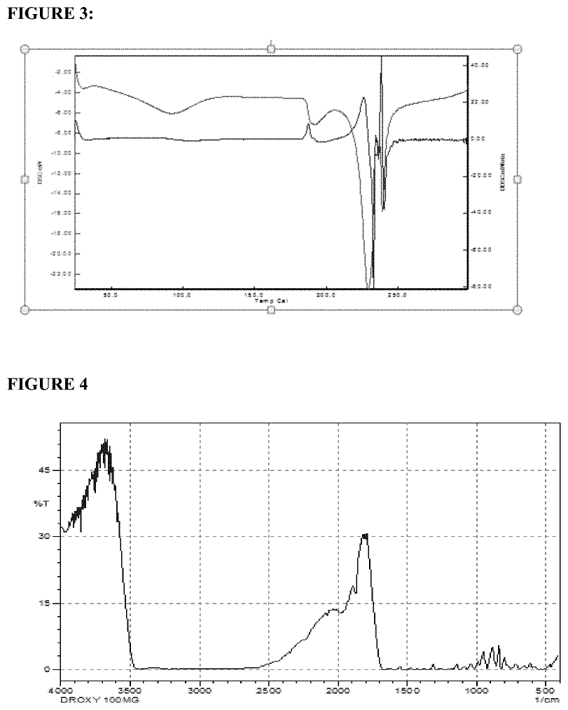

DSC thermograms of pure Droxidopa, HPMC K 200M and Droxidopa with HPMC K200M (FIGS. 1, 2, 3) were obtained with DSC refrigerated cooling system (Model Q1000, TA instruments, UK). Samples (0.8-6.3 mg) were weighed and transferred into the equipment for analysis in sealed hermetically aluminum pans and heated with continuous purge of argon. The instrument was calibrated with sapphire and indium before running the samples. Thermal behavior of the samples was investigated at a scanning rate of 10° C./min, from 0° C. to 300° C.

Result in reference to FIGS. 1, 2, and 3DSC was performed to characterize thermal changes in the melting behavior of Droxidopa with other excipients present in different formulations. The thermograms of heat verses temperature for pure Droxidopa drug were recorded. Endothermic peaks of Droxidopa pure drug were observed at 230° C. It shows that there was no interaction between drug and polymers.

Fourier Transform Infrared Spectroscopy (Ftir) Studies

The KBr disk sample preparation technique (pressed pellet technique) was used to obtain the IR spectra of the samples on an IR spectrophotometer. The infrared spectra of pure drug, HPMC K 200M, and mixture of Droxidopa and HPMC K 200 M were recorded by using a Fourier transform infrared spectrophotometer. A base line correction was made using dried potassium bromide and then the spectrum of the pure drug. Approximately 100 mg of potassium bromide (spectroscopic grade) was thoroughly mixed with approximately one mg of test sample in a glass mortar. The mixture was compressed into transparent disks in a moisture free atmosphere and IR spectra were obtained. The scanning range was selected between 4000 and 400 cm⁻¹. The obtained spectra were compared with those reported in official compendia. Characteristic peaks attributable to functional groups present in molecule of each drug were assigned to establish the identity.

If there is any interaction between drug and excipients, the characteristic peak corresponding to any functional group of pure drug sample will be absent or shifted to other frequency region, in IR spectra of prepared formulations.

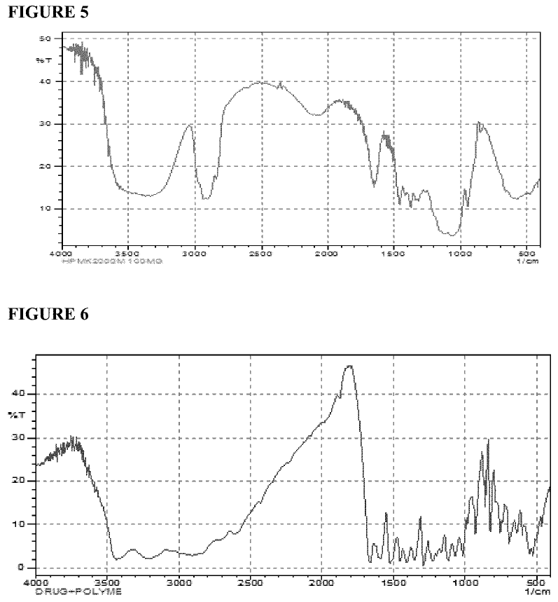

Results are illustrated in The FT-IR spectra's of Droxidopa and were shown in FIGS. 4, 5, and 6. The main absorption bands of drug were observed O—H stretching at 3539 cm⁻¹, C═O at 2349 cm⁻¹, NH₂ at 3500 cm⁻¹, C═C at 1620 cm⁻¹, C—H stretch at 3000 cm⁻¹ were present in spectra that indicating compatibility. It shows that there was no significant change in the chemical integrity of the drug.

Evaluation of Droxidopa Sustained Release Floating Tablets

A. Precompression Parameters of Droxidopa Formulations:

All the formulations were evaluated for precompression studies such as bulk density, tapped density, angle of repose, compressibility index and hausners ratio as shown in the Table No 5, the results indicates that they have good flow properties. From the results it was observed that the values obtained of all formulations were within acceptable limits as per I.P standards.

TABLE 5
Precompressionparameters of the Droxidopa Formulations.

| | Angle of | Bulk density | Tappeddensity | Carr's Index | Hausner's |
|---|---|---|---|---|---|
| Formulation | repose (θ) | (g/cm³) | (g/cm³) | (%) | ratio |
| F1 | 25.32 ± 0.52 | 0.53 ± 0.01 | 0.83 ± 0.03 | 17.73 ± 0.24 | 1.12 ± 0.10 |
| F2 | 27.12 ± 0.39 | 0.59 ± 0.03 | 0.91 ± 0.05 | 18.44 ± 0.31 | 1.12 ± 0.12 |
| F3 | 26.38 ± 0.71 | 0.61 ± 0.05 | 0.68 ± 0.07 | 10.67 ± 0.50 | 1.24 ± 0.08 |
| F4 | 28.96 ± 0.62 | 0.67 ± 0.01 | 0.91 ± 0.09 | 16.29 ± 0.71 | 1.36 ± 0.04 |
| F5 | 29.15 ± 0.73 | 0.72 ± 0.05 | 0.89 ± 0.04 | 15.54 ± 0.23 | 1.28 ± 0.03 |
| F6 | 28.72 ± 0.54 | 0.81 ± 0.05 | 0.73 ± 0.03 | 19.97 ± 0.45 | 1.21 ± 0.09 |
| F7 | 25.56 ± 0.33 | 0.77 ± 0.03 | 0.89 ± 0.01 | 11.64 ± 0.75 | 1.36 ± 0.03 |
| F8 | 25.67 ± 0.42 | 0.61 ± 0.07 | 0.91 ± 0.02 | 13.39 ± 0.41 | 1.32 ± 0.04 |
| F9 | 26.23 ± 0.41 | 0.66 ± 0.09 | 0.98 ± 0.01 | 13.12 ± 0.49 | 1.41 ± 0.09 |
| F11 | 25.59 ± 0.52 | 0.75 ± 0.01 | 0.84 ± 0.02 | 10.71 ± 0.22 | 1.12 ± 0.03 |
| F12 | 24.54 ± 0.31 | 0.61 ± 0.03 | 0.77 ± 0.01 | 20.77 ± 0.47 | 1.26 ± 0.09 |
| F13 | 29.05 ± 0.43 | 0.62 ± 0.05 | 0.75 ± 0.04 | 17.33 ± 0.26 | 1.20 ± 0.10 |
| F14 | 29.97 ± 0.41 | 0.50 ± 0.06 | 0.63 ± 0.02 | 20.63 ± 0.47 | 1.26 ± 0.91 |
| F15 | 28.15 ± 0.71 | 0.69 ± 0.03 | 0.78 ± 0.05 | 11.53 ± 0.19 | 1.30 ± 0.04 |
| F16 | 21.55 ± 0.81 | 0.68 ± 0.05 | 0.79 ± 0.04 | 13.92 ± 0.37 | 1.16 ± 0.19 |
| F17 | 33.56 ± 0.32 | 0.53 ± 0.04 | 0.65 ± 0.03 | 18.46 ± 0.28 | 1.22 ± 0.08 |
| F18 | 29.13 ± 0.33 | 0.74 ± 0.04 | 0.86 ± 0.02 | 13.95 ± 0.54 | 1.16 ± 0.04 |

Note:
All values are expressed as mean ± SD, n = 3

B. Post Compression Parameters of Droxidopa Formulations:

Post compression parameters includes determination of weight variation, friability, hardness, thickness and drug content. All these tests were performed according to IP standards.

TABLE 6
Post Compression parameters ofDroxidopa Formulations

| | Weight | | | | |
|---|---|---|---|---|---|
| | Variation | Hardness | Thickness | Friability | Drug Content |
| Formulation | (mg)*** | (kg/cm²)* | (mm)* | (%)** | (%)** |
| F1 | 590 ± 1.00 | 4.12 ± 0.91 | 3.50 ± 0.08 | 0.12 ± 0.17 | 100.41 ± 0.84 |
| F2 | 585 ± 1.65 | 4.20 ± 0.20 | 3.85 ± 0.12 | 0.28 ± 0.16 | 100.15 ± 0.53 |
| F3 | 580 ± 1.45 | 4.23 ± 0.82 | 3.91 ± 0.07 | 0.34 ± 0.08 | 98.54 ± 0.75 |
| F4 | 595 ± 1.32 | 4.34 ± 0.41 | 3.45 ± 0.05 | 0.26 ± 0.87 | 92.12 ± 0.67 |
| F5 | 580 ± 1.56 | 4.39 ± 0.72 | 3.87 ± 0.09 | 0.23 ± 0.91 | 90.54 ± 0.15 |
| F6 | 585 ± 1.23 | 4.42 ± 0.39 | 3.63 ± 0.13 | 0.54 ± 0.02 | 91.63 ± 0.36 |
| F7 | 610 ± 1.94 | 4.51 ± 0.71 | 3.21 ± 0.15 | 0.11 ± 0.20 | 96.32 ± 0.61 |
| F8 | 605 ± 1.89 | 4.11 ± 0.61 | 3.50 ± 0.10 | 0.29 ± 0.12 | 99.58 ± 0.66 |
| F9 | 600 ± 1.32 | 4.12 ± 0.89 | 3.98 ± 0.19 | 0.27 ± 0.17 | 98.63 ± 0.39 |
| F11 | 608 ± 1.75 | 4.79 ± 0.65 | 4.29 ± 0.12 | 0.15 ± 0.02 | 101.75 ± 0.50 |
| F12 | 606 ± 1.88 | 5.56 ± 0.01 | 4.31 ± 0.01 | 0.29 ± 0.08 | 99.15 ± 0.38 |
| F13 | 600 ± 1.00 | 6.00 ± 0.07 | 4.50 ± 0.02 | 0.51 ± 0.18 | 100.52 ± 0.15 |
| F14 | 611 ± 2.50 | 5.85 ± 0.05 | 4.46 ± 0.07 | 0.20 ± 0.17 | 98.50 ± 0.85 |
| F15 | 611 ± 1.20 | 5.60 ± 0.05 | 4.40 ± 0.05 | 0.30 ± 0.15 | 102.58 ± 0.76 |
| F16 | 612 ± 3.65 | 6.24 ± 0.17 | 4.20 ± 0.05 | 0.35 ± 0.18 | 99.60 ± 0.10 |
| F17 | 712 ± 1.36 | 6.28 ± 0.37 | 4.43 ± 0.06 | 0.42 ± 0.35 | 98.45 ± 1.60 |
| F18 | 708 ± 2.16 | 6.28 ± 0.05 | 4.29 ± 0.07 | 0.52 ± 0.29 | 100.75 ± 0.13 |

*All values are expressed as mean ± SD, n = 5;
**All values are expressed as mean ± SD, n = 10;
***All values are expressed as mean ± SD, n = 20;

Result: The post compression parameters such as weight variation, friability, hardness, drug concentrate shown in the Table No. 6. From the results it was observed that the values obtained of all formulations were within acceptable limits as per I.P standards.

C. IN VITRO FLOATING OR BUOYANCY STUDIES: The time taken for tablet to emerge on the surface of the medium is called the floating lag time (FLT) or buoyancy lag time (BLT) and duration of time the dosage form constantly remains on the surface of the medium is called the total floating time (TFT). The in vitro floating behavior of the tablets was studied in USP type II dissolution apparatus at 37±0.5° C. in 900 ml of simulated gastric fluid at 0.1N HCl. The time required for the tablet to emerge on the surface is floating lag time (FLT) or buoyancy lag time (BLT). The time of duration of floatation was observed visually.

TABLE 7
Floating lag time and total floating
time of Droxidopa floating tablets

| | Floatinglag | Buoyancy |
|---|---|---|
| Formulation | time (sec) | time (hrs) |
| F1 | 10 ± 0.12 | 5 |
| F2 | 10 ± 0.19 | 6 |
| F3 | 10 ± 0.11 | 6 |
| F4 | 10 ± 0.13 | 7 |
| F5 | 10 ± 0.13 | 7 |
| F6 | 10 ± 0.15 | 7 |
| F7 | 10 ± 0.21 | 8 |
| F8 | 10 ± 0.23 | 8 |
| F9 | 10 ± 0.19 | 8 |
| F11 | 20 ± 0.21 | >12 |
| F12 | 25 ± 0.23 | >12 |
| F13 | 25 ± 0.14 | >12 |
| F14 | 30 ± 0.13 | >12 |
| F15 | 55 ± 0.11 | >12 |
| F16 | 60 ± 0.17 | >12 |
| F17 | 60 ± 0.21 | >12 |
| F18 | 60 ± 0.24 | >12 |

Note:
All the values are expressed as mean ± SD. (n = 3)

D. In Vitro Dissolution Studies:

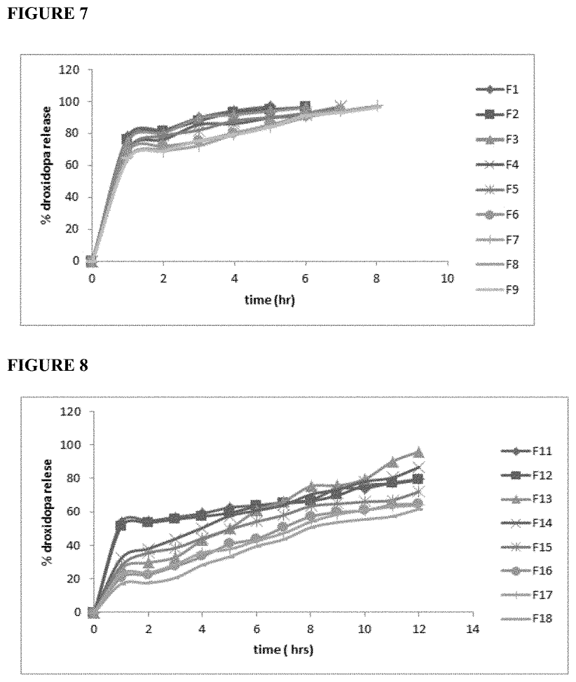

In vitro dissolution studies of the Droxidopa floating tablets were performed in USP Type II dissolution apparatus. 900 ml of 0.1N HCl was used as dissolution media and temperature was maintained at 37° C.±0.5° C. with paddle rotation speed at 50 rpm. About 5 ml of aliquot were withdrawn at 1, 2, 3, 4, 5, 6, 7, 8, 9, 10, 11 and 12 hrs and replaced with 5 ml of dissolution medium to maintain the sink condition. Samples were filtered through whatman filter paper and analysed UV spectrophotometrically at 280 nm. Results are displayed in Table 8. In vitro dissolution studies of F1-F9 is displayed in FIGS. 7 and F11-18 in FIG. 8.

TABLE 8
In vitro dissolution studies of Droxidopa sustained release floating tablets (F1-F9)

| Time | % Droxidopa release ( Mean ± SD, n = 3 ) | | | | | | | | |
|---|---|---|---|---|---|---|---|---|---|
| (hrs) | F1 | F2 | F3 | F4 | F5 | F6 | F7 | F8 | F9 |
| 1 | 78.2 ± 0.68 | 76.3 ± 1.52 | 75.4 ± 1.23 | 70.1 ± 0.75 | 70.0 ± 0.96 | 68.6 ± 1.23 | 65.2 ± 0.76 | 64.2 ± 1.56 | 64.0 ± 0.87 |
| 2 | 82.0 ± 0.97 | 81.1 ± 0.89 | 80.2 ± 1.34 | 76.0 ± 0.68 | 78.4 ± 0.99 | 71.8 ± 1.36 | 69.4 ± 0.54 | 70.3 ± 1.64 | 69.1 ± 0.75 |
| 3 | 89.8 ± 1.68 | 88.0 ± 0.95 | 89.6 ± 1.42 | 82.5 ± 0.63 | 82.0 ± 0.54 | 75.0 ± 0.96 | 72.3 ± 0.65 | 75.0 ± 1.78 | 74.6 ± 0.88 |
| 4 | 94.2 ± 0.74 | 93.0 ± 0.67 | 91.7 ± 1.57 | 86.0 ± 0.71 | 87.9 ± 0.82 | 80.0 ± 0.65 | 79.4 ± 0.98 | 80.1 ± 1.99 | 79.0 ± 0.75 |
| 5 | 96.9 ± 0.23 | 95.1 ± 0.24 | 93.9 ± 1.95 | 89.7 ± 1.23 | 90.1 ± 0.91 | 85.5 ± 0.61 | 84.0 ± 0.12 | 85.2 ± 1.94 | 84.2 ± 0.43 |
| 6 | — | 96.4 ± 0.31 | 96.0 ± 1.6 | 91.3 ± 1.84 | 92.7 ± 0.75 | 90.6 ± 0.87 | 90.2 ± 1.13 | 91.3 ± 1.56 | 90.9 ± 0.72 |
| 7 | — | — | — | 96.5 ± 1.56 | 96.9 ± 0.66 | 95.0 ± 0.61 | 94.2 ± 0.45 | 94.3 ± 1.23 | 93.0 ± 0.80 |
| 8 | — | — | — | — | — | — | 97.2 ± 0.56 | 96.9 ± 0.99 | 96.1 ± 1.20 |

TABLE 9
In vitro dissolution studies of droxidopa sustained release floating tablets (F11-F18)

| Time | | | % Droxidopa Release ( Mean ± SD, n = 3 ) | | | | | |
|---|---|---|---|---|---|---|---|---|
| (hr) | F11 | F12 | F13 | F14 | F15 | F16 | F17 | F18 |
| 1 | 53.8 ± 1.89 | 51.4 ± 1.00 | 26.3 ± 0.55 | 32.3 ± 0.83 | 27.9 ± 0.87 | 20.9 ± 0.65 | 22.9 ± 1.00 | 17.1 ± 0.66 |
| 2 | 54.6 ± 1.57 | 53.3 ± 0.89 | 29.7 ± 0.83 | 38.0 ± 0.48 | 35.5 ± 0.36 | 22.6 ± 0.36 | 23.7 ± 1.12 | 17.5 ± 0.69 |
| 3 | 56.9 ± 0.79 | 55.6 ± 0.78 | 32.9 ± 1.00 | 43.9 ± 0.65 | 38.4 ± 0.76 | 27.5 ± 0.97 | 28.6 ± 2.63 | 20.6 ± 1.65 |
| 4 | 59.0 ± 0.54 | 57.6 ± 0.54 | 43.2 ± 0.65 | 50.5 ± 0.18 | 44.3 ± 0.65 | 33.4 ± 0.45 | 35.7 ± 2.03 | 28.4 ± 2.21 |
| 5 | 62.6 ± 0.38 | 59.4 ± 1.02 | 50.0 ± 0.76 | 57.6 ± 0.52 | 49.6 ± 0.86 | 40.7 ± 0.58 | 37.7 ± 0.56 | 32.2 ± 2.06 |
| 6 | 63.9 ± 1.87 | 63.2 ± 0.36 | 60.7 ± 0.55 | 60.8 ± 0.39 | 54.2 ± 1.85 | 43.8 ± 0.69 | 42.8 ± 1.32 | 39.5 ± 0.88 |
| 7 | 65.7 ± 2.75 | 65.1 ± 0.86 | 66.3 ± 0.36 | 64.1 ± 0.90 | 58.0 ± 2.76 | 50.8 ± 1.67 | 47.3 ± 0.87 | 43.6 ± 0.98 |
| 8 | 67.2 ± 1.76 | 66.4 ± 0.65 | 75.2 ± 0.69 | 70.4 ± 0.73 | 63.4 ± 1.55 | 57.2 ± 1.69 | 54.1 ± 2.52 | 50.7 ± 1.85 |
| 9 | 73.8 ± 0.56 | 69.9 ± 0.55 | 79.6 ± 0.95 | 73.4 ± 0.66 | 64.8 ± 1.23 | 59.8 ± 2.65 | 58.5 ± 2.13 | 53.8 ± 1.96 |
| 10 | 74.2 ± 1.85 | 75.7 ± 0.65 | 85.2 ± 1.20 | 78.1 ± 0.95 | 66.0 ± 0.89 | 60.8 ± 2.02 | 61.0 ± 1.42 | 55.6 ± 1.98 |
| 11 | 77.3 ± 2.67 | 76.8 ± 0.89 | 92.1 ± 0.98 | 80.3 ± 0.56 | 67.0 ± 0.79 | 64.1 ± 1.95 | 63.0 ± 1.35 | 57.3 ± 1.87 |
| 12 | 79.6 ± 1.38 | 79.0 ± 1.00 | 96.0 ± 1.36 | 86.7 ± 1.06 | 72.1 ± 0.62 | 64.7 ± 0.82 | 63.9 ± 0.64 | 61.8 ± 2.09 |

E. Drug Release Kinetics

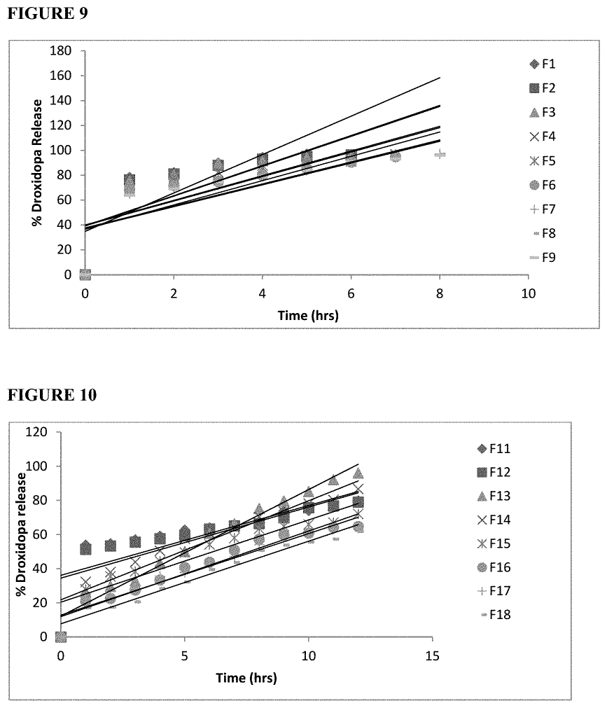

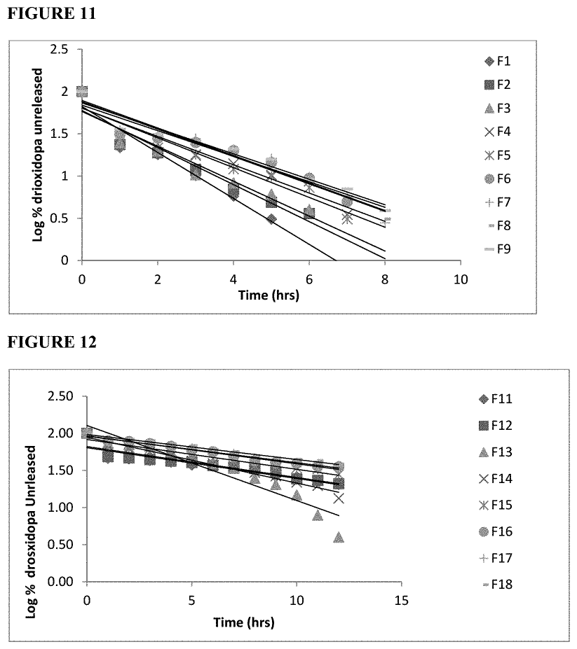

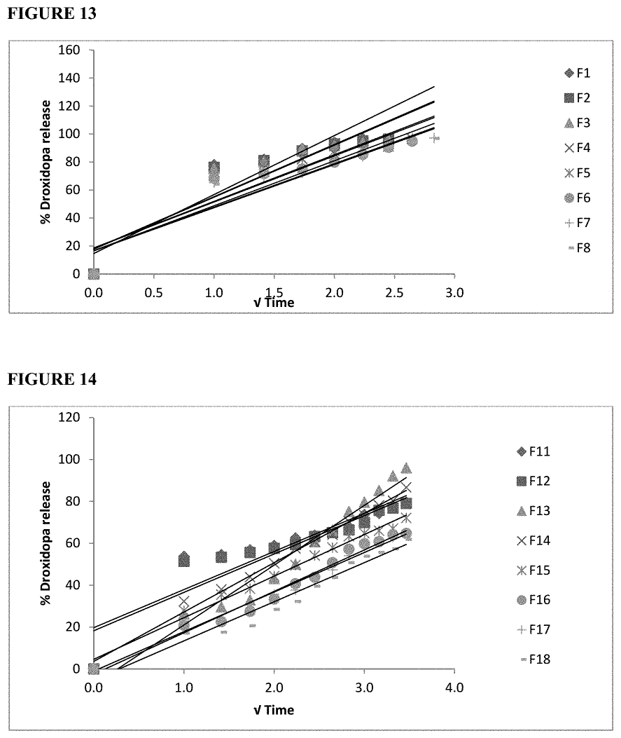

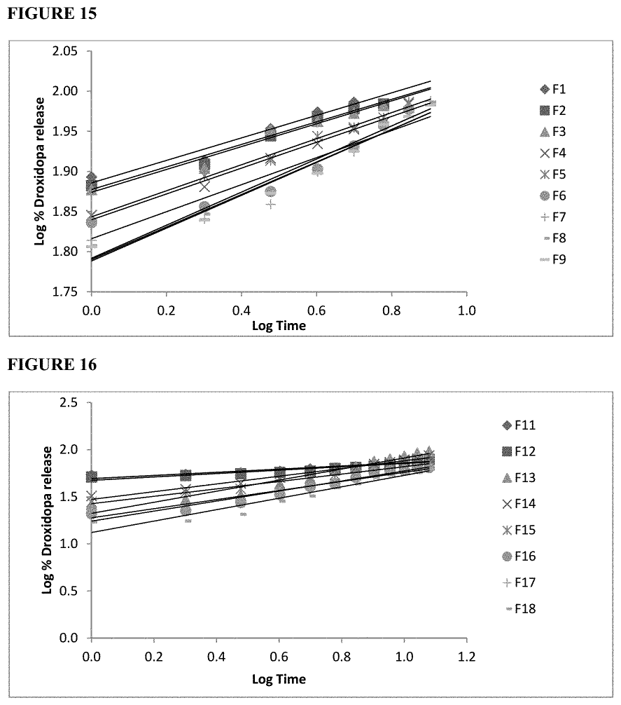

The in vitro drug release kinetic studies of all the formulations (F1 to F9 and F11 to F18) were fitted in various kinetic models like Zero order, First order, Higuchi and Korsmeyer-Peppas shown in FIGS. 9 to 16 respectively and Table No. 10.

TABLE 10
Release kinetics of droxidopa sustained release floating tablets
(F1 to F9 and F11 to F18):

| | Droxidopa release Kinetics | | | | | | | | |
|---|---|---|---|---|---|---|---|---|---|
| | Order | | Kinetic Models | | | Correlation Coefficient Values | | | |
| | Zero | First | Higuchi | Korsmeyerp | | (r) | | | |
| Formulation | K₀ | K₁ | K_{H} | ppas | T_{1/2} | Zero | First | | Korsmeyerp |
| code | (M/s) | (hr⁻¹) | (M/s) | N | hr | Order | Order | Higuchi | ppas |
| F1 | 15.43 | 0.624 | 42.11 | 0.140 | 1.11 | 0.618 | 0.940 | 0.865 | 0.955 |
| F2 | 12.11 | 0.502 | 37.35 | 0.140 | 1.38 | 0.585 | 0.931 | 0.843 | 0.976 |
| F3 | 12.01 | 0.474 | 37.12 | 0.142 | 1.46 | 0.582 | 0.922 | 0.841 | 0.966 |
| F4 | 9.834 | 0.385 | 32.96 | 0.161 | 1.80 | 0.600 | 0.918 | 0.850 | 0.986 |
| F5 | 9.901 | 0.426 | 33.27 | 0.162 | 1.63 | 0.598 | 0.939 | 0.849 | 0.992 |
| F6 | 9.775 | 0.348 | 32.13 | 0.168 | 1.99 | 0.635 | 0.914 | 0.865 | 0.900 |
| F7 | 8.825 | 0.373 | 30.86 | 0.202 | 1.86 | 0.678 | 0.939 | 0.893 | 0.928 |
| F8 | 8.806 | 0.368 | 30.98 | 0.206 | 1.88 | 0.669 | 0.952 | 0.891 | 0.968 |
| F9 | 8.743 | 0.345 | 30.73 | 0.204 | 2.01 | 0.671 | 0.953 | 0.892 | 0.961 |
| F11 | 4.806 | 0.094 | 18.16 | 0.166 | 7.34 | 0.625 | 0.842 | 0.831 | 0.871 |
| F12 | 4.165 | 0.094 | 18.34 | 0.181 | 7.34 | 0.656 | 0.860 | 0.851 | 0.878 |
| F13 | 7.405 | 0.233 | 28.53 | 0.588 | 2.98 | 0.973 | 0.904 | 0.968 | 0.946 |
| F14 | 5.801 | 0.140 | 23.54 | 0.410 | 4.93 | 0.898 | 0.974 | 0.992 | 0.983 |
| F15 | 4.814 | 0.092 | 19.86 | 0.392 | 7.52 | 0.865 | 0.960 | 0.986 | 0.987 |
| F16 | 5.010 | 0.085 | 19.77 | 0.532 | 8.13 | 0.940 | 0.980 | 0.981 | 0.955 |
| F17 | 4.780 | 0.081 | 18.90 | 0.482 | 8.60 | 0.938 | 0.979 | 0.981 | 0.944 |
| F18 | 4.826 | 0.076 | 18.65 | 0.606 | 9.12 | 0.965 | 0.986 | 0.966 | 0.933 |

The in vitro drug release kinetic studies of all the formulations (F1 to F9 and F11 to F18) were fitted in various kinetic models like Zero order, First order, Higuchi and Korsmeyer-Peppas shown in FIG. 9 to 16 and Table No. 10. The results indicated that, except optimized formulation (F13) the drug release of all formulations follows first order release kinetics due to high correlation coefficient values of first order kinetics.

F. Stability Studies:

The optimized tablet formulation F13, was subjected to accelerated stability studies according to ICH guidelines at 40° C./75% RH for 6 months and Room temperature.

TABLE 11
STABILITY STUDIES FOR OPTIMIZED FORMULATION

| | Optimized Formulation F13 | |
|---|---|---|
| No. of | | % Drug release after 12 hrs |

| Months | Room Temp. | 40° C./75% RH | % Drug content |
|---|---|---|---|
| 0 | 96.03 ± 0.35 | 96.03 ± 0.15 | 100.50 ± 0.05 |
| 1 | 96.01 ± 0.22 | 96.04 ± 1.30 | 100.32 ± 0.15 |
| 3 | 95.97 ± 0.56 | 95.98 ± 0.54 | 100.26 ± 0.09 |
| 6 | 95.68 ± 0.75 | 95.69 ± 0.86 | 100.15 ± 1.09 |

Stability studies was conducted on tablets of formulation F13 as per ICH guidelines. No significant changes were observed in any of the studied parameters during the study period (6 months), thus it could be concluded that formulation was stable.

Discussion

The present invention aimed to formulate and evaluate Droxidopa sustained release floating tablets. These floating tablets were formulated by using semi synthetic polymers as rate retarding agent. They produce better patient compliance, maintain uniform drug therapeutic level, are cost-effective, have broad regulatory acceptance and increase the safety margin for high-potency therapeutic agents. Pre formulation studies were carried out and all parameters were found within the specification limits. Different formulations of Droxidopa were formulated by using different concentrations of the polymer and evaluated for pre and post compression parameters. Various formulations of Droxidopa floating tablets were prepared by using various viscosity grades of hydroxyl propyl methyl cellulose polymers such as HPMC K 15M, HPMC K50M, HPMC K 100M and HPMC K200M, optimum concentration of NaHCO₃ and citric acid are gas generating agents.

Tablets float when sodium bicarbonate reacts with acidic buffer (0.1N HCl) and forms carbon dioxide gas which is entrapped by gel formed polymer resulting in a decrease in density of tablet. Shorter floating lag time and longer floating time are considered to be good floating properties.

Microcrystalline cellulose as diluent was found to be beneficial in improving the drug release rate and floating properties are used in different proportions by direct compression technique. The tablets showed floating lag time (<1 min) and remained buoyant for more than 12 hours. The floating lag time was found to be significantly increasing with increase in concentration of polymer.

In vitro release studies indicate that, Cumulative percent drug release of the formulations containing HPMC K200M polymer of F11 to F18 in 12 h were found to be in the range of 61.82% to 96.02% (figure. 10). Formulation F13 containing (15%) 90 mg of HPMC K200M polymer had better dissolution profile along with desired sustained action as compared to other formulations and showed promising dissolution parameters (t50%=5 h, and t90%=10.65 h) with desired floating properties. From the result it was clear that the retarding rate was higher for formulations containing HPMC K200M compared with low viscous grades of HPMC K15M, K50M and K100M formulations. This may be owing to the drug may have entrapped within a polymer matrix causing a decrease in the rate of drug release.

All the formulations were subjected to various drug release kinetic studies. The results indicated that drug release from optimized formulation F13, shows zero order release and the mechanism of drug release was found to be diffusion.

Higuchi's equation was found to be 0.968, which shows that the data fitted well to Higuchi's square root of time equation confirming the release followed diffusion mechanism. To confirm the diffusion mechanism, the data were fitted into Korsmeyer-Peppas equation, the formulations showed good linearity (r=0.946), indicating that diffusion was the predominant mechanism of drug release from the formulation. The value of release exponent (n) for the above formulations are 0.588, which indicates anomalous transport mechanism (coupling of the diffusion and erosion mechanism). Overall, the present invention shows that viscosity is a major factor affecting the Droxidopa drug release and floating properties of floating drug delivery system. From the dissolution study it was observed that F13 containing HPMC K200M polymer showed the lowest release rate compared with others, due to its high molecular weight and viscosity.

Stability studies was conducted on tablets of formulation F13 as per ICH guidelines. No significant changes were observed in any of the studied parameters during the study period (6 months), thus it could be concluded that formulation was stable.

The sustained release of Droxidopa floating tablets were prepared by simple and cost effective technique for the treatment of orthostatic hypotension, increases the patient compliance and effectiveness of therapy.

I claim:

1. A floating sustained release tablet formulation of Droxidopa, comprising:
Droxidopa—200-600 mg;
Polymer—10-25% by weight (60-150 mg);
additives comprising diluent—110-120 mg;
effervescent agent—70-80 mg;
gas generating agent—30-50 mg;
glidant—10 mg; and
lubricant—10 mg,
wherein the total tablet weight ranges from 590 to 610 mg and the sustained release tablet is a gastro-retentive tablet that releases Droxidopa in sustained manner for a period of 10-12 hours.

2. The floating sustained release tablet formulation of Droxidopa as claimed in claim 1, wherein the polymer comprises of Cellulose hydroxypropyl methyl ether.

3. The floating sustained release tablet formulation of Droxidopa as claimed in claim 2, wherein the cellulose hydroxyl propyl methyl ether is hydroxyl propyl methyl cellulose HPMC K200M.

4. The floating sustained release tablet formulation of Droxidopa as claimed in claim 1, wherein the diluent is Microcrystalline cellulose.

5. The floating sustained release tablet formulation of Droxidopa as claimed in claim 1, wherein the gas generating agent is sodium bicarbonate.

6. The floating sustained release tablet formulation of Droxidopa as claimed in claim 1, wherein the effervescent agent is anhydrous citric acid or tartaric acid.

7. The floating sustained release tablet formulation of Droxidopa as claimed in claim 1, wherein the glidant is talc and lubricant is Magnesium stearate.

8. The floating sustained release tablet formulation of Droxidopa as claimed in claim 1, wherein floating lag time of the tabled is less than 60 seconds and buoyancy time of over 12 hrs.

9. The floating sustained release tablet formulation of Droxidopa as claimed in claim 1, wherein the tablet formulation dosage is twice a day.

10. A method of preparation of floating sustained release tablet formulation of Droxidopa as claimed in claim 1, comprising steps:
accurately weighing Droxidopa—200-400 mg, polymer HPMCK200M (90-100 mg), sodium bicarbonate 50 mg, citric acid—75 mg, micro crystalline cellulose-115 mg and blending;
addition of lubricant-10 mg of magnesium stearate and glidant −10 mg talc added as post lubricant and further mixed for additional 2-3 minutes;
sieving of ingredients by passing through 40# sieves; and′ punching of gastro retentive tablets using the rotary tablet compression machine by direct compression technique using 12 mm punches, and
wherein the tablets are buoyant on gastric fluid and release Droxidopa for sustained period of over 12 hrs.
